(19) United States
(12) Patent Application Publication
(10) Pub. No.: US 2023/0292755 A1
(43) Pub. Date: Sep. 21, 2023

(54) USE OF A GLYCINE BETAINE DERIVATIVE AS A PEDICULICIDAL AGENT

(71) Applicant: SURFACTGREEN, COMPIEGNE CEDEX (FR)

(72) Inventors: FRANCIS GALLE, RENNES CEDEX 7 (FR); XAVIER ROUSSEL, LE MANS (FR); FREDDY PESSEL, RENNES CEDEX 7 (FR); CÉDRIC BOYERE, RENNES (FR)

(21) Appl. No.: 18/017,094

(22) PCT Filed: Jul. 16, 2021
(86) PCT No.: PCT/FR2021/051324

§ 371 Date: Jan. 20, 2023

(30) Foreign Application Priority Data
Jul. 22, 2020 (FR) FR2007689

(51) Int. Cl. A01N 37/44; A01P 7/04

(52) U.S. Cl. CPC A01N 37/44; A01P 7/04

(57) ABSTRACT

The invention relates to a surfactant composition containing at least one ester or amide salt of glycine betaine, or a dermatological composition containing it, for use as a pediculicidal agent by topical application on the hair and the scalp. It also relates to a method for preventing and/or treating infestations of head lice and/or nits, comprising the application to a fabric surface of a topical composition containing a surfactant composition as defined above.

### SUBJECT MATTER OF THE INVENTION

A subject matter of the invention is a surfactant composition including at least one glycine betaine ester or amide salt, or a topical composition containing it, for its use as pediculicidal agent. Another subject matter of the invention is a method for preventing and/or treating infestations by head lice and/or nits, comprising the application, to a surface to be treated, in particular to the hair and the scalp, of a topical composition including a surfactant composition as defined above.

### BACKGROUND OF THE INVENTION

Head lice are a scourge which remains difficult to eradicate despite the diversity of the treatments available on the market. In the two usual places of transmission, which are a community of children and the family, contamination is above all interhuman and takes place directly by contact, even brief, of a parasitized subject with another subject, whatever his/her socioeconomic origin. Once contaminated, the subject is quickly infested, since the female lays from 4 to 10 eggs or nits per day for 3 to 4 weeks, each nit forming, after 7 to 9 days, a larva which becomes an adult approximately one week later. Unlike the body louse (Pediculus humanus var corporis), the head louse (Pediculus humanus var capitis) does not transmit pathogenic agents. It nevertheless causes itching, essentially in the nape of the neck, and around the ears, due to the presence of allergenic substances in the saliva of the parasites.

The following are listed among the methods used to combat lice:
chemical treatments based on a pediculicide acting on lice and possibly also on nits, such as organophosphorus compounds, including malathion, organochlorine compounds, such as lindane, and also pyrethrins and synthetic pyrethroids, including permethrin, d-phenothrin and depallethrin, which are optionally used in combination with piperonyl butoxide,
mechanical treatments, using specific combs,
physical means, using compositions of fatty substances, such as vegetable or silicone oils, squalane or also fatty acid esters, which obstruct the respiratory tract of the lice, resulting in their death.

However, these methods exhibit several disadvantages. This is because the chemical treatments based on organophosphorus or organochlorine compounds can pose toxicity problems which in particular prevent them from being used by pregnant women or children. In addition, the effectiveness of pediculicides has declined in recent years due to the fact that lice have begun to develop resistance to these compounds. Finally, their environmental impact is criticized.

For their part, the physical and mechanical treatments do not exhibit an adverse effect on the health or the appearance of resistance, but they are often not sufficiently effective, in particular on nits, and require their application to be repeated at regular intervals. Above all, the effectiveness of the physical treatments requires a leave-on time of at least eight hours. For all these reasons, the result is an often insufficient compliance with the treatment, resulting in reinfestation. Furthermore, the oils used in this application tend to make the hair greasy and make these products difficult to rinse out, which discourages some consumers from using them.

In this context, it remains necessary to have available a new pediculicidal composition which does not present problems of toxicity to humans and the environment and which makes it possible to exterminate lice efficiently, typically in a single application and after a reduced leave-on time.

The applicant company has now discovered, unexpectedly and surprisingly, that certain biodegradable cationic surfactants based on glycine betaine derivatives exhibit advantageous pediculicidal properties. It has in particular been demonstrated that these compounds make it possible to eradicate at least 90%, indeed even 100%, of the lice, in a few minutes, typically in less than 15 minutes, indeed even in 10 minutes. These compounds furthermore exhibit a solubility in water which makes it possible to formulate them easily and to rinse them easily.

It has already been suggested that surfactants could exhibit pediculicidal properties. The patent FR 2 996 453 thus discloses the use, as pediculicide, of a combination of nonionic and anionic surfactants. However, the effectiveness of this mixture remains moderate, since only 55% of the lice are eliminated after 30 minutes, unless colloidal silica particles are added. Furthermore, it is not demonstrated that cationic surfactants, a fortiori based on glycine betaine derivatives, might exhibit the same effectiveness.

Cationic surfactants derived from glycine betaine, consisting of glycine betaine amide and ester salts, have furthermore been described for their use in insecticidal compositions (EP 3 584 303), optionally in combination with nonionic surfactants of alkyl polyglycoside type (WO 2005/121294). However, it is not specifically suggested that they be used in compositions intended for the treatment of lice, or that these surfactants may themselves exhibit pediculicidal properties.

### SUMMARY OF THE INVENTION

A subject matter of the invention is a surfactant composition including at least one glycine betaine derivative of formula (1): [(CH₃)₃N⁺—CH₂—COZ—R]ₙXⁿ⁻, where Z denotes an oxygen atom or an —NH group, R is a saturated or unsaturated, linear or branched, alkyl group comprising from 8 to 24 carbon atoms, X is an organic or inorganic anion and n has the value 1 or 2, or a topical composition containing it, for its use as pediculicidal agent.

Another subject matter of the invention is a method for preventing and/or treating infestations by head lice and/or nits, comprising the application, to a surface to be treated, in particular to the hair and the scalp, of a topical composition including a surfactant composition as defined above.

A further subject matter of the invention is the use of this surfactant composition, or of a topical composition comprising it, for eliminating head lice and/or nits.

### DETAILED DESCRIPTION

The present invention relates to a surfactant composition including at least one glycine betaine derivative, which is an glycine betaine ester or amide salt, for its use as pediculicidal agent, that is to say as medicament for topical application in the prevention and/or the treatment of head lice and/or nits. These two types of glycine betaine derivatives, and also their processes of preparation, will now be described in more detail.

### Glycine Betaine Ester Salts

Glycine betaine ester salts can be obtained following a process comprising the successive stages consisting in:
(1) reacting glycine betaine or one of its salts with at least one saturated or unsaturated, linear or branched, fatty alcohol including from 8 to 24 carbon atoms, in the presence of an organic or inorganic acid;
(2) cooling the reaction medium to a temperature of 20 to 90° C.; and
(3) recovering the surfactant composition thus obtained.

The first stage of this process consists in esterifying glycine betaine, or trimethylglycine. The glycine betaine can be of vegetable or synthetic origin. It is necessary to protonate it beforehand using an organic or inorganic acid, insofar as it exists in the zwitterionic form (presence of a carboxylate function). The acid can in particular be chosen from inorganic acids, such as hydrochloric acid, sulfuric acid, perhydric acids, such as perchloric acid, and their mixtures. In an alternative form, it can be chosen from organic acids, such as alkyl sulfuric acids, for example decyl or lauryl sulfuric acid; arylsulfonic acids, such as benzenesulfonic acid or para-toluenesulfonic acid; alkylsulfonic acids, such as triflic acid, methanesulfonic acid, ethanesulfonic acid, decylsulfonic acid, laurylsulfonic acid or camphorsulfonic acid; sulfosuccinic acid; and their mixtures. Lewis acids can also be used. Preferably, it is an alkylsulfonic acid and in particular methanesulfonic acid, insofar as it is easily biodegradable.

During the esterification, the acid function of the salified betaine is reacted with a fatty alcohol, to result in a glycine betaine ester in salt form. The term “fatty alcohol” is understood to mean a saturated or unsaturated, linear or branched (preferably linear), alcohol comprising from 8 to 24 carbon atoms. Examples of such fatty alcohols can be chosen from the group consisting of: caprylyl alcohol (C8:0), octan-2-ol, decyl alcohol (C10:0), undecyl alcohol (C11:0), lauryl alcohol (C12:0), myristyl alcohol (C14:0), cetyl alcohol (C16:0), palmitoleyl alcohol (C16:1), stearyl alcohol (C18:0), oleyl alcohol (C18:1), linoleyl alcohol (C18:2), linolenyl alcohol (C18:3), arachidyl alcohol (C20:0), arachidonyl alcohol (C20:4), behenyl alcohol (C22:0), 2-hexyldecanol, 2-octyldodecanol, 2-decyltetradecanol and their mixtures. Mixtures of fatty alcohols which can be used can be produced from one or more vegetable oils and in particular from soybean, olive, sunflower, corn, palm, copra, cottonseed, linseed, wheat germ, safflower or rapeseed oil, for example.

The esterification reaction is generally carried out in the absence of solvent. The water produced during the reaction furthermore contributes to the dissolution of the glycine betaine in the reaction mixture.

For the implementation of this reaction, it is possible, for example, to use from 0.8 to 6.0 equivalents, preferably from 0.8 to 2 equivalents, for example from 0.9 to 1.0 equivalent, or in an alternative form from 1.1 to 1.8 equivalents, preferentially in this case from 1.2 to 1.6 equivalents and better still from 1.3 to 1.5 equivalents of fatty alcohol or, in a second alternative form, from 4.0 to 6.0 equivalents, preferentially in this case from 4.5 to 5.5 equivalents and better still from 4.8 to 5.2 equivalents of fatty alcohol.

In addition, use is advantageously made of 1.01 to 3.0 molar equivalents, preferably of 1.5 to 2.0 molar equivalents, for example of 1.5 to 1.9 molar equivalents, and preferentially of 1.5 to 1.7 molar equivalents of organic or inorganic acid, or in an alternative form of 1.02 to 1.08 molar equivalents, preferentially in this case of 1.03 to 1.07 molar equivalents and better still of 1.04 to 1.06 molar equivalents of organic or inorganic acid per 1 equivalent of glycine betaine. The esterification is carried out at a temperature ranging, for example, from 120 to 180° C., preferably from 150 to 180° C. The reaction can be carried out under atmospheric pressure or preferably under reduced pressure, for example at a pressure of 10 to 600 mbar. The pressure will generally become lower as the chain length of the fatty alcohol involved increases. The reaction medium is subsequently cooled to a temperature of 20 to 90° C.

The surfactant composition thus obtained is then recovered, which composition includes at least one glycine betaine ester salt of formula [(CH₃)₃N⁺—CH₂—COOR]ₙXⁿ⁻, where: X is an organic or inorganic anion, R is an alkyl radical corresponding to the fatty alcohol R—OH employed in the esterification reaction, and n has the value 1 or 2.

The anion X results from the acid employed in the first stage of the process and can thus in particular be a chloride, a sulfate, a perchlorate, an alkyl sulfate ion, in particular a decyl sulfate or lauryl sulfate ion, an arylsulfonate ion, in particular a benzenesulfonate or para-toluenesulfonate ion, an alkylsulfonate ion, in particular a triflate, methanesulfonate, ethanesulfonate, decylsulfonate, laurylsulfonate or camphorsulfonate ion, or a sulfosuccinate ion. It is preferred according to the invention for X to be chosen from the alkylsulfonates and the arylsulfonates, in particular from the methanesulfonate, ethanesulfonate, triflate, para-toluenesulfonate and camphorsulfonate ions. It is advantageously the methanesulfonate ion.

The radical R can for its part be chosen from the octyl (C8:0), 1-methylheptyl (C8r:0), decyl (C10:0), undecyl (C11:0), lauryl (C12:0), myristyl (C14:0), cetyl (C16:0), palmitoleyl (C16:1), stearyl (C18:0), oleyl (C18:1), linoleyl (C18:2), linolenyl (C18:3), arachidyl (C20:0), arachidonyl (C20:4), behenyl (C22:0), 2-hexyldecyl, 2-octyldodecyl and 2-decyltetradecyl groups.

It is clearly understood that, in the case where several fatty alcohols are employed in the esterification reaction, the surfactant composition obtained according to the invention will comprise several glycine betaine ester salts. The term “a glycine betaine ester salt” should thus be understood, in the context of this description and unless otherwise indicated, as referring to one or more of these salts.

The process described above makes it possible more specifically to obtain a surfactant composition including, and generally consisting of, the following constituents:
(a) at least one glycine betaine ester salt of formula (1): [(CH₃)₃N⁺—CH₂—COO—R]ₙXⁿ⁻, where R is a saturated or unsaturated, linear or branched, alkyl group comprising from 8 to 24 carbon atoms,
(b) at least one fatty alcohol of formula R—OH,
(c) an organic or inorganic acid of formula HX,
(d) a glycine betaine salt of formula [(CH₃)₃N⁺—CH₂—COOH]ₙXⁿ⁻, and
(e) optionally, at least one dialkyl ether of formula R—O—R, where X is an organic or inorganic anion and n has the value 1 or 2.

This surfactant composition can be used as is in the present invention. In this case, it generally includes from 15% to 85% by weight of glycine betaine ester salt.

In a first alternative form, the surfactant composition includes:
(a) from 65% to 85% by weight, preferably from 70% to 80% by weight, of glycine betaine ester salt,
(b) from 1% to 20% by weight, for example from 1% to 9% by weight or from 10% to 20% by weight, of fatty alcohol,
(c) from 1% to 20% by weight, for example from 5% to 15% by weight, of organic or inorganic acid,
(d) from 1% to 20% by weight, for example from 2% to 15% by weight, of glycine betaine salt,
(e) from 0% to 15% by weight, for example from 2% to 10% by weight, of dialkyl ether.

In a second alternative form, which is preferred, the surfactant composition includes: (a) from 15% to 50% by weight, preferably from 20% to 30% by weight, more preferentially from 25% to 30% by weight, of glycine betaine ester salt,
(b) from 50% to 70% by weight, for example from 60% to 65% or from 65% to 70% by weight, of fatty alcohol,
(c) from 0% to 5% by weight, for example from 0% to 1% by weight, of organic or inorganic acid,
(d) from 0% to 3% by weight, for example from 0% to 1% by weight, of glycine betaine salt,
(e) from 0% to 15% by weight, for example from 2% to 10% by weight, of dialkyl ether.

Advantageously, the surfactant composition does not include a constituent other than components (a) to (e) above, that is to say that the total of the constituents (a) to (e) is equal to 100%. In an alternative form, the above process can include an additional stage consisting in isolating the glycine betaine ester salt present in this composition, which can be used as is in the present invention. In the latter case, the surfactant composition used according to the invention will comprise at least 90%, preferably at least 95%, indeed even at least 99%, by weight of glycine betaine derivative.

### Glycine Betaine Amide Salts

These glycine betaine derivatives can be prepared following a process comprising the successive stages consisting in:
(1) reacting the glycine betaine or one of its salts with a saturated or unsaturated, linear or branched, C₄-C₈ alcohol in the presence of an organic or inorganic acid, at a temperature ranging, for example, from 100 to 180° C. and under reduced pressure;
(2) cooling the reaction medium to a temperature of 20 to 80° C.;
(3) adding one or more alkylamines including from 8 to 24 carbon atoms;
(4) removing the residual alcohol; and
(5) recovering the surfactant composition thus obtained.

The first stage of this process consists of an esterification reaction of the glycine betaine, which can be carried out in a similar way to the production of the glycine betaine esters, except that use is made of one or more linear and/or branched C₄-C₈ alcohol(s) in the presence of the acid, which can be chosen from those described above. Examples of such alcohols comprise butanol, pentanol, 3-methylbutan-1-ol (or isoamyl alcohol), fusel alcohol (mixture of pentanol, 2-methylbutan-1-ol and 3-methylbutan-1-ol), hexanol, heptanol, octanol and their mixtures. The term “butanol” is understood to mean equally well in this description n-butanol, isobutanol and sec-butanol. Butanol, and more particularly n-butanol, is preferred for use in this invention. This reaction is generally carried out in the absence of any solvent, the alcohol used constituting both the reactant and the medium. The water produced during the reaction also contributes to the dissolution of the glycine betaine in the reaction mixture. Use may generally be made of from 1.1 to 20 equivalents, for example from 2 to 4 equivalents, of linear or branched C₄-C₈ alcohol and of from 1.0 to 1.5 equivalents of sulfonic acid, for example from 1.0 to 1.2 equivalents and preferentially 1.1 equivalents of sulfonic acid, per 1 equivalent of glycine betaine. The esterification can be carried out at a temperature of 100 to 180° C., preferentially of 100 to 160° C., more preferentially of 120 to 150° C. or of 130 to 160° C., at atmospheric pressure or under reduced pressure.

The product of the esterification reaction may or may not be treated so as to separate the glycine betaine ester salt formed from the reaction medium. To do this, it is possible, for example, to carry out a filtration of the reaction medium, which makes it possible to separate the abovementioned salified ester, which is soluble in the alcohol, from the other constituents, which are not soluble.

One or more C₈-C₂₄ alkylamine(s) is/are subsequently added, either to the reaction medium or to the isolated ester. Examples of such amines are: dodecylamine or laurylamine, tetradecylamine, hexadecylamine, octadecylamine, docosanylamine, eicosanylamine and their mixtures.

In this stage, the alkylamine is advantageously used in the molten form. The amount of alkylamine(s) added can, for example, represent from 0.9 to 1.5 equivalents and preferably from 1.0 to 1.2 equivalents, per 1 equivalent of glycine betaine initially employed. This aminolysis reaction is typically carried out at a temperature of 50 to 180° C. and preferably of 120 to 140° C., under reduced pressure, for example under a pressure of 1 to 30 mbar. In parallel with the aminolysis reaction, the alcohol is removed by distillation under reduced pressure. The aminolysis reaction and the distillation take place over a period of time of 1 to 7 hours, in particular of 3 to 5 hours.

The surfactant composition thus obtained is then recovered.

This process makes it possible to obtain a surfactant composition comprising, and preferably consisting of:
(a) one or more glycine betaine amide salt(s) of formula (1): [(CH₃)₃N⁺—CH₂—CONH—R]ₙXⁿ⁻, where R is a saturated or unsaturated, linear or branched, alkyl group comprising from 8 to 24 carbon atoms;
(b) one or more alkylammonium salt(s) of formula (2): [NH₃⁺R]ₙXⁿ⁻, where R is a saturated or unsaturated, linear or branched, alkyl group comprising from 8 to 24 carbon atoms;
(c) one or more glycine betaine ester salt(s) of formula (3): [(CH₃)₃N⁺—CH₂—COOR′]ₙXⁿ⁻, where R′ is a saturated or unsaturated, linear or branched, alkyl radical containing from 4 to 8 carbon atoms; and
(d) glycine betaine of formula (4): (CH₃)₃N⁺—CH₂—COO⁻;
where X is an organic or inorganic anion and n has the value 1 or 2.

This surfactant composition can be used as is in the present invention. In this case, it generally includes from 60% to 98% by weight, for example from 70% to 80% by weight, of glycine betaine amide salt. The constituent (b) can represent from 0% to 25% by weight, for example from 15% to 20% by weight, the constituent (c) from 0% to 15% by weight, for example from 5% to 10% by weight, and the constituent (d) from 0% to 5% by weight, with respect to the total weight of the surfactant composition. Advantageously, the latter does not include a constituent other than the components (a) to (d) above. In an alternative form, the above process can include an additional stage consisting in isolating the glycine betaine amide salt present in this composition, which can be used as is in the present invention. In the latter case, the surfactant composition used according to the invention will comprise at least 90%, preferably at least 95%, indeed even at least 99%, by weight of glycine betaine derivative.

### Topical Composition

The surfactant composition described above can itself be used as pediculicidal agent, optionally after dilution in water to reach a dry matter content of 0.5% to 20% by weight, for example, preferably of 1% to 10% by weight and, better still, of 3% to 5% by weight. In a preferred embodiment of the invention, the surfactant composition can be mixed with different ingredients to form a composition denoted in the present description by “topical composition”, insofar as it exists in the form of a product suitable for topical application to the surface to be treated, such as the hair and the scalp, in particular in the form of a lotion, a gel or a cream. It is generally an aqueous composition or an emulsion of the oil-in-water (O/W), water-in-oil (W/O) or multiple (for example W/O/W) type. In the case where the topical composition is formulated in the form of an emulsion, it is preferred for it to be an oil-in-water emulsion. In all cases, the topical composition generally includes from 0.5% to 20% by weight, preferably from 1% to 10% by weight and better still from 3% to 5% by weight of glycine betaine derivative.

This topical composition can in particular be packaged in a tube, a bottle, a pump-action spray or a jar. In an alternative form, it can be packaged in an aerosol container, in order to ensure application of the composition in vaporized form. In the latter case, the topical composition preferably comprises at least one propellant.

The topical composition used according to the invention generally constitutes a cosmetic or dermatological composition and in this case respectively comprises a cosmetically or dermatologically acceptable medium, that is to say a medium compatible with the hair and the scalp and not generating unacceptable irritation of the scalp or other adverse effect after application to the hair.

The topical composition generally includes an aqueous phase comprising water and optionally one or more cosmetically acceptable water-soluble solvents chosen from C₁-C₄ alcohols, such as ethanol, isopropanol, tert-butanol or n-butanol, polyols, such as glycerol, propylene glycol and polyethylene glycols, and their mixtures. Preferably, the composition has a total water content of between 5% and 95% by weight, preferably between 10% and 90% by weight, for example between 40% and 85% by weight, in particular between 50% and 80% by weight, with respect to the total weight of the composition. The pH of this composition generally varies from 3 to 9, preferably from 3 to 7, preferentially from 5 to 6.

It can additionally comprise at least one fatty phase including at least one fatty substance, so as to form an emulsion. Preferably, the fatty substance or substances are chosen from oils. The term “oils” is understood to mean a compound which is liquid at ambient temperature (25° C.) and atmospheric pressure (10⁵ Pa) and which, when it is introduced in a proportion of at least 1% by weight into water at 25° C., is not at all soluble in the water, or soluble to the extent of less than 10% by weight, with respect to the weight of oil introduced into the water.

Mention may in particular be made, as examples of oils, of fatty alcohols, fatty esters, hydrocarbons of vegetable or mineral origin, triglycerides and the vegetable oils containing them, and their mixtures. Mention may in particular be made, as fatty alcohols, of branched and/or unsaturated C₁₀-C₂₀ fatty alcohols, such as octyldodecanol and oleyl alcohol. Examples of fatty esters are the esters of acids and of monoalcohol chosen from: mono- and polyesters of saturated linear C₂-C₁₀ (preferably C₆-C₁₀) acids and of saturated linear C₁₀-C₁₈ (preferably C₁₀-C₁₄) monoalcohols, mono- and polyesters of saturated linear C₁₀-C₂₀ acids and of branched or unsaturated C₃-C₂₀ (preferably C₃-C₁₀) monoalcohols; mono- and polyesters of branched or unsaturated C₅-C₂₀ acids and of branched or unsaturated C₅-C₂₀ monoalcohols; mono- and polyesters of branched or unsaturated C₅-C₂₀ acids and of linear C₂-C₄ monoalcohols. Examples of such fatty esters are in particular the coco-caprate/caprylate mixture, ethyl macadamiate, the ethyl ester of shea butter, isostearyl isostearate, isononyl isononanoate, ethylhexyl isononanoate, hexyl neopentanoate, ethylhexyl neopentanoate, isostearyl neopentanoate, isodecyl neopentanoate, isopropyl myristate, octyldodecyl myristate, isopropyl palmitate, ethylhexyl palmitate, hexyl laurate, isoamyl laurate, cetostearyl nonanoate, propylheptyl caprylate, diisopropyl adipate, diethylhexyl adipate, diisopropyl sebacate and diisoamyl sebacate.

Mention may be made, as hydrocarbons, of squalane (C₃₀), in particular vegetable squalane extracted from olive oil, and hemisqualane (C₁₅). Examples of triglycerides are triglycerides of C₆-C₁₂ fatty acids, such as triglycerides of caprylic and capric acids, and triheptanoin. Examples of vegetable oils are in particular wheat germ, sunflower, argan, hibiscus, coriander, grape seed, sesame, corn, apricot, castor, shea, avocado, olive, soybean, sweet almond, palm, rapeseed, cottonseed, hazelnut, macadamia, jojoba, alfalfa, poppy, red kuri squash, pumpkin, blackcurrant, evening primrose, lavender, borage, millet, barley, quinoa, rye, safflower, candlenut, passionflower, muscat rose, echium, camelina or camellia oil.

The fatty substances can represent from 1% to 30% by weight, with respect to the total weight of the composition.

The topical composition used according to the invention can also comprise at least one ordinary excipient, chosen in particular from nonionic surfactants, cationic surfactants other than the glycine betaine derivatives, anionic surfactants, amphoteric surfactants; antioxidants; pearlescent and/or opacifying agents; pigments; fillers; sequestering agents; thickeners; nonthickening polymers, such as aminosilicones and/or cationic polymers; fragrances; preservatives; and their mixtures.

Preferably, the anionic surfactants are chosen from alkylcarbonylisethionic acid salts, such as those identified under the INCI names SODIUM COCOYL ISETHIONATE and SODIUM COCOYL METHYL ISETHIONATE; lactylic acid salts, such as SODIUM LAUROYL LACTYLATE; salts of N-acylated amino acids, such as SODIUM LAUROYL GLYCINATE, SODIUM LAUROYL SARCOSINATE, SODIUM LAUROYL TAURATE and SODIUM OLIVOYL GLUTAMATE; sulfated anionic surfactants, chosen in particular from alkyl sulfate salts, in particular SODIUM COCO SULFATE and POTASSIUM LAURYL SULFATE, C₈-C₁₄ alkyl ether sulfate salts, such as SODIUM LAURYL ETHER SULFATE; soaps in the form of carboxylic acid salts, in particular SODIUM OLIVATE and SODIUM PALMITATE; and alkyl ether carboxylic surfactants, such as lauryl ether carboxylic acids or sodium lauryl ether carboxylates.

The nonionic surfactant(s) used in the cosmetic composition are preferentially chosen from: saturated or unsaturated, linear or branched, C₈ to C₄₀ alcohols etherified with 1 to 100 moles of ethylene oxide, preferably from 2 to 50, more particularly from 2 to 40, moles of ethylene oxide, preferably comprising one or two fatty chains; oxyethylenated vegetable oils, which are or are not saturated, comprising from 1 to 100, preferably from 2 to 50, moles of ethylene oxide; sucrose esters, such as sucrose stearate and sucrose distearate, mono- or polyglycerolated C₈ to C₄₀ alcohols comprising from 1 to 50 moles of glycerol, preferably from 1 to 10 moles of glycerol; amides of saturated or unsaturated, linear or branched, C₈ to C₃₀ fatty acids, of poly(ethylene oxide) or of mono- or diethanol, in particular cocamide MEA or coconut fatty acid monoethanolamide; esters of saturated or unsaturated, linear or branched, C₈ to C₃₀ acids and of polyethylene glycols; esters of saturated or unsaturated, linear or branched, C₈ to C₃₀ acids and of sorbitol, which are preferably oxyethylenated; and their mixtures.

The amphoteric surfactant(s) used in the cosmetic composition used in the present invention can in particular be derivatives of secondary or tertiary aliphatic amines, which are optionally quaternized, in which the aliphatic group is a linear or branched chain comprising from 8 to 22 carbon atoms, said amine derivatives containing at least one anionic group, such as, for example, a carboxylate, sulfonate, sulfate, phosphate or phosphonate group. Mention may in particular be made of (C₈-C₂₀)alkyl betaines, (C₈-C₂₀)alkyl sulfobetaines, (C₈-C₂₀)alkylamido(C₃-C₈)alkyl betaines, such as cocamidopropyl betaine, and (C₈-C₂₀)alkylamido(C₆-C₈)alkyl sulfobetaines.

The cationic surfactant(s) optionally used in addition to the glycine betaine derivatives can be chosen from salts of primary, secondary or tertiary fatty amines, which are optionally polyoxyalkylenated, quaternary ammonium salts, and their mixtures.

The thickener(s) can be chosen from cellulose thickening agents, for example hydroxyethylcellulose, hydroxypropylcellulose and carboxymethylcellulose, guar gum and its derivatives, for example hydroxypropyl guar, gums of microbial origin, such as xanthan gum and scleroglucan gum, synthetic thickening agents, such as crosslinked homopolymers of acrylic acid or of acrylamidopropanesulfonic acid, nonionic, anionic, cationic or amphoteric associative polymers. Mention may more particularly be made, among the cationic polymers which can be used as thickening polymers, of polymers of the polyamine, polyaminoamide and polyquaternary ammonium type, in particular cationic celluloses, cationic guar gums and homopolymers or copolymers of dimethyldiallylammonium halides.

It is preferred for the topical composition according to the invention not to include a neurotoxic pediculicidal active ingredient. This term denotes a molecule which, after having crossed the cuticle of the louse, acts as a neurotoxin. Such is in particular the case with pyrethrins and pyrethroids, which act by keeping the voltage-gated sodium channels open for an abnormally long time, which leads to spastic paralysis and the death of the louse, and also malathion, which acts as acetylcholinesterase inhibitor, thus leading to blockage of nerve impulses and buildup of the neurotransmitter, which results in fatal spastic paralysis. On the other hand, the composition according to the invention may or may not include one or more compounds capable of killing the lice by asphyxiating them and/or by dehydrating them by dissolving their exoskeleton. Examples of such compounds are silicone oils, vegetable oils, in particular coconut oil, and fatty acid esters.

The topical composition can, alternatively or in addition, include one or more repellents chosen, for example, from linalool-containing essential oils, such as essential oils of lavender, of lavandin or of tea tree, which exhibit a repellent action on lice.

### Process/Use

The invention relates to a method for preventing and/or treating infestations by head lice and/or nits, comprising the topical application, to a surface to be treated, in particular to the hair and the scalp, of a topical composition including a surfactant composition as defined above. A further subject matter of the invention is the use of this surfactant composition, or of a topical composition comprising it, for eliminating head lice and/or nits.

The use of this surfactant composition for the preparation of a topical composition intended to prevent and/or combat infestations by head lice and/or nits is also described here.

The topical composition used according to the invention can be applied to any surface to be treated, in particular to surfaces made of fabric, for example of seats or of bedding, and/or to body surfaces, more particularly to the hair or the scalp. In this particular application, the topical composition can be applied to dry or wet hair, and preferably to dry hair. According to one embodiment, the method according to the invention consists in applying, to the hair and the scalp, an effective amount of the topical composition, by spraying or manual application, in kneading the hair and/or distributing the composition over the hair using a comb, in leaving the composition to stand on the hair for a period of time ranging from 5 minutes to one hour, preferably from 5 minutes to 30 minutes, more preferentially from 5 minutes to 15 minutes, and in then rinsing the hair with water or using a shampoo. A lice comb can be used before or after rinsing the composition in order to remove dead insects and possibly their nits.

This treatment can be repeated one to three times if necessary, with a time interval ranging from 1 day to 1 month between each application.

### EXAMPLES

A better understanding of the invention will be obtained in the light of the following examples, which are given purely by way of illustration without the aim of limiting the scope of the invention, defined by the appended claims.

### Example 1: Synthesis of a Surfactant Composition Based on Glycine Betaine Ester Salt (GBE C12)

Glycine betaine (1.0 eq) and lauryl alcohol (0.9 eq) are introduced into a reactor. The setpoint temperature in the mixture is fixed at 170° C. and the pressure is reduced down to a value of 60 mbar. Once the pressure and temperature setpoints have been reached, a 70% methanesulfonic acid solution (1.6 eq) is added to the reaction mixture. As soon as the addition is complete, the setpoint temperature is brought back to 150° C. and the pressure is maintained at a value of 30 mbar. Five hours after the start of the introduction of the acid, the reaction mixture is allowed to cool to 80° C., then the product is recovered, cooled down to ambient temperature, and constitutes the surfactant composition according to the invention, which includes the following constituents:

| | Composition by weight |
|---|---|
| Lauryl betainate mesylate | 66.5% |
| Glycine betaine mesylate | 17.1% |
| Lauryl alcohol | 2.6% |
| Methanesulfonic acid | 10.6% |
| Dilauryl ether | 3.2% |

### Example 2: Synthesis of a Surfactant Composition Based on Glycine Betaine Amide Salt (GBA C12)

Glycine betaine (1.0 eq) and hexanol (3.0 eq) are introduced into a reactor surmounted by a Dean & Stark apparatus filled with hexanol. A pressure-equalizing dropping funnel containing a 70% methanesulfonic acid solution (1.1 eq) is fixed to the top of the reactor. The mixture is stirred and heated to 150° C. under reduced pressure at 600 mbar. Once the reaction conditions have been reached, the 70% methanesulfonic acid solution is gradually introduced into the reaction mixture. Once the addition is complete, the pressure is steadily reduced until it reaches 300 mbar in order to accelerate the removal of the water and to make possible the displacement of the equilibrium toward the glycine betaine ester. The degree of conversion is monitored by ¹H NMR analyses.

The NMR method consists in producing a ¹H spectrum of the sample dissolved in a CDCl₃/CD₃OD (1/1, v/v) mixture, taking the signal of methanol at 3.31 ppm as reference. The signals characteristic of the different compounds are subsequently integrated: GBOC6MsO (4.35 ppm, s, 2H), GBMsO (4.28 ppm, s, 2H), hexanol (3.53 ppm, s, 2H), methanesulfonate (2.74 ppm, s, 3H), dihexyl ether (3.40 ppm, t, 4H), where GBOC6XO denotes the sulfonate salt of the glycine betaine ester formed and GBXO denotes the glycine betaine sulfonate formed. The signal characteristic of methanesulfonate takes into account both the methanesulfonic acid present in the medium, and also the methanesulfonate which is the counterion of the glycine betaine and of the hexyl betainate mesylate (GBOC6MsO).

The degree of conversion of the reaction is obtained by virtue of the integration values by the following calculation:

$$η = \frac{{I}_{GBOC 6 MsO} / 2}{{I}_{GBMsO} / 2 + {I}_{GBOC 6 MsO} / 2} = \frac{{I}_{GBOC 6 MsO}}{{I}_{GBMsO} + {I}_{GBOC 6 MsO}}$$

where:
η is the degree of conversion
Iᵢ is the integration value of the signal characteristic of the compound i.

Once the degree of conversion of the esterification reaction reaches 96%, the reaction mixture is allowed to cool down to 60° C. During this cooling phase, the Dean & Stark assembly is replaced by a distillation assembly and the reactor is placed under reduced pressure in order to remove a part of the hexanol and the remaining traces of water in the reaction mixture. Once the mixture is at 80° C., premelted laurylamine (1.1 eq) is added. The reaction mixture is then heated to 150° C. under reduced pressure. The pressure is gradually decreased down to 10 mbar. After the complete distillation of the hexanol (approximately 4 hours), the reaction mixture is recovered and constitutes the surfactant composition.

The latter exhibits the following composition by weight:

| | Composition by weight |
|---|---|
| Betainylaminododecane mesylate | 73.9% |
| Laurylammonium mesylate | 14.2% |
| Hexyl betainate mesylate | 7.7% |
| Glycine betaine | 1.0% |
| Hexanol | 1.0% |
| Dihexyl ether | 2.2% |

### Example 3: Efficacy Test

The surfactant compositions prepared as described in examples 1 and 2 above were tested following the recommendations of the ECHA relating to the evaluation of the efficacy of biocidal products (Guidance on the Biocidal Products Regulation Volume II Efficacy—Assessment and Evaluation (Parts B+C), Version 3.0 April 2018).

These compositions were thus each sprayed, in the form of aqueous solutions at 3% by weight of dry matter, into 4 boxes each including 25 lice (Pediculus humanus capitis), then the percentage of mortality was evaluated after 5, 10 and 15 min. The mean of the results obtained in the 4 boxes was calculated. The results of these tests are presented in table 3 below.

| | Mortality rate (%) | |
|---|---|---|
| Time | GBE C12 | GBA C12 |
| 5 min | 94% | 77% |
| 10 min | 100% | 100% |
| 15 min | 100% | 100% |

This example thus demonstrates that the surfactant compositions according to the invention make it possible to eliminate all of the lice after only 10 minutes of application.

### Example 4: Comparative Example

The test presented in example 3 was reproduced using one and the same concentration of different cationic surfactants instead of the glycine betaine derivatives according to the invention.

The results of this comparative test are collated in table 4 below:

| | Mortality rate (%) | | |
|---|---|---|---|
| | Benzalkonium | Cetrimonium | Dodecyltrimethylammonium |
| Time | chloride | chloride | chloride |
| 5 min | 70 | 79 | 49 |
| 10 min | 80 | 94 | 78 |
| 15 min | 82 | 100 | 91 |

Conventional cationic surfactants thus prove to be less effective than the compounds according to the invention. In addition, the time required to eliminate 100% of the lice is 60 min in the case of benzalkonium chloride and 30 min in the case of dodecyltrimethylammonium chloride.

### Example 5: Anti-Lice Compositions

The products below can be prepared using the surfactant compositions according to the invention, based respectively on glycine betaine ester or amide salts or on their mixtures, identified below by “Glycine betaine derivative”. The other constituents of these products are identified by their chemical names (in lower case) or their INCI names (in upper case).

Anti-lice shampoo

| Components | % by weight |
|---|---|
| COCAMIDOPROPYL BETAINE | 20% |
| COCAMIDE MEA | 5% |
| Glycine betaine derivative | 3% |
| Sodium chloride | 1% |
| TOCOPHERYL ACETATE | 0.50% |
| COCO NUCIFERA OIL | 0.50% |
| BENZYL ALCOHOL | 0.60% |
| Potassium sorbate | 0.30% |
| Citric acid | q.s. for pH |
| | 5.5 |
| Water | q.s. for |
| | 100% |

Anti-lice shampoo

| Components | % by weight |
|---|---|
| COCAMIDOPROPYL BETAINE | 24% |
| Glycine betaine derivative | 3% |
| TOCOPHERYL ACETATE | 0.50% |
| GLYCERYL OLEATE | 0.50% |
| BENZYL ALCOHOL | 0.60% |
| POTASSIUM SORBATE | 0.30% |
| TRISODIUM CITRATE | q.s. for pH |
| | 5.5 |
| Water | q.s. for |
| | 100% |

Anti-lice lotion

| Components | % by weight |
|---|---|
| Glycine betaine derivative | 3 |
| Glycerol | 3 |
| Propylene glycol | 2 |
| Water | q.s. for 100% |

Anti-lice shampoo

| Components | % by weight |
|---|---|
| COCAMIDOPROPYL | 5 |
| BETAINE | |
| Glycine betaine derivative | 3 |
| Glycerol | 3 |
| NaCl | 2.5 |
| Sodium citrate | q.s. for pH = 5.5 |
| Propylene glycol | 2 |
| Water | q.s. for 100% |

Anti-lice cream

| Components | % by weight |
|---|---|
| Sunflower oil | 10 |
| Stearyl alcohol | 6 |
| SPAN 60 | 3.9 |
| Glycine betaine | 3 |
| derivative | |
| Glycerol | 3 |
| Polysorbate 80 | 1.1 |
| Hydroxyethylcellulose | 0.5 |
| Citric acid (4%) | q.s. for pH = 5.5 |
| Water | q.s. for 100% |

1-10. (canceled)

11. A method for preventing and/or treating infestations by head lice and/or nits, comprising the application, to a surface to be treated, of a surfactant composition including at least one glycine betaine derivative of formula (1): [(CH₃)₃N⁺—CH₂—COZ—R]ₙXⁿ⁻, where Z denotes an oxygen atom or an —NH group, R is a saturated or unsaturated, linear or branched, alkyl group comprising from 8 to 24 carbon atoms, X is an organic or inorganic anion and n has the value 1 or 2, or a composition containing the same.

12. The method of claim 11, wherein the radical R is selected from the group consisting of octyl (C8:0), 1-methylheptyl (C8r:0), decyl (C10:0), undecyl (C11:0), lauryl (C12:0), myristyl (C14:0), cetyl (C16:0), palmitoleyl (C16:1), stearyl (C18:0), oleyl (C18:1), linoleyl (C18:2), linolenyl (C18:3), arachidyl (C20:0), arachidonyl (C20:4), behenyl (C22:0), 2-hexyldecyl, 2-octyldodecyl and 2-decyltetradecyl groups.

13. The method of claim 11, wherein the anion X is selected from the group consisting of a chloride, a sulfate, a perchlorate, an alkyl sulfate ion, an arylsulfonate ion, an alkylsulfonate ion and a sulfosuccinate ion.

14. The method of claim 13, wherein the alkyl sulfate ion is a decyl sulfate or lauryl sulfate ion.

15. The method of claim 13, wherein the arylsulfonate ion is a benzenesulfonate or para-toluenesulfonate ion.

16. The method of claim 13, wherein the alkylsulfonate ion is a triflate, methanesulfonate, ethanesulfonate, decylsulfonate, laurylsulfonate or camphorsulfonate ion.

17. The method of claim 11, wherein the surfactant composition includes the following constituents:
(a) at least one glycine betaine ester salt of formula (1): [(CH₃)₃N⁺—CH₂—COO—R]ₙXⁿ⁻, where R is a saturated or unsaturated, linear or branched, alkyl group comprising from 8 to 24 carbon atoms,
(b) at least one fatty alcohol of formula R—OH where R is selected from the group consisting of octyl (C8:0), 1-methylheptyl (C8r:0), decyl (C10:0), undecyl (C11:0), lauryl (C12:0), myristyl (C14:0), cetyl (C16:0), palmitoleyl (C16:1), stearyl (C18:0), oleyl (C18:1), linoleyl (C18:2), linolenyl (C18:3), arachidyl (C20:0), arachidonyl (C20:4), behenyl (C22:0), 2-hexyldecyl, 2-octyldodecyl and 2-decyltetradecyl groups,
(c) an organic or inorganic acid of formula HX, and
(d) a glycine betaine salt of formula [(CH₃)₃N⁺—CH₂—COOH]ₙXⁿ⁻,
where X is an organic or inorganic anion and n has the value 1 or 2.

18. The method of claim 17, wherein the surfactant composition consists of the claimed constituents.

19. The method of claim 17, wherein the surfactant composition includes:
(a) from 65% to 85% by weight, of glycine betaine ester salt,
(b) from 1% to 20% by weight, of fatty alcohol,
(c) from 1% to 20% by weight of organic or inorganic acid,
(d) from 1% to 20% by weight, of glycine betaine salt,
(e) from 0% to 15% by weight, of dialkyl ether.

20. The method of claim 17, wherein the surfactant composition includes:
(a) from 15% to 50% by weight of glycine betaine ester salt,
(b) from 50% to 70% by weight, of fatty alcohol,
(c) from 0% to 5% by weight, of organic or inorganic acid,
(d) from 0% to 3% by weight, of glycine betaine salt,
(e) from 0% to 15% by weight, of dialkyl ether.

21. The method of claim 11, wherein the surfactant composition includes the following constituents:
(a) one or more glycine betaine amide salt(s) of formula (1): [(CH₃)₃N⁺—CH₂—CONH—R]ₙXⁿ⁻, where R is a saturated or unsaturated, linear or branched, alkyl group comprising from 8 to 24 carbon atoms;
(b) one or more alkylammonium salt(s) of formula (2): [NH₃⁺R]ₙXⁿ⁻, where R is a saturated or unsaturated, linear or branched, alkyl group comprising from 8 to 24 carbon atoms;
(c) one or more glycine betaine ester salt(s) of formula (3): [(CH₃)₃N⁺—CH₂—COOR′]ₙXⁿ⁻, where R′ is a saturated or unsaturated, linear or branched, alkyl radical containing from 4 to 8 carbon atoms; and
(d) glycine betaine of formula (4): (CH₃)₃N⁺—CH₂—COO⁻;
where X is an organic or inorganic anion and n has the value 1 or 2.

22. The method of claim 21, wherein the surfactant composition consists of the claimed constituents.

23. The method of claim 11, wherein the composition is applied to the hair and the scalp in an effective amount, by spraying or manual application, the hair is kneaded and/or the composition is distributed over the hair using a comb, the composition is left to stand on the hair for a period of time ranging from 5 minutes to one hour and then the hair is rinsed with water or using a shampoo.

24. The method of claim 11, wherein the surface is made of fabric.

25. The method of claim 11, wherein the surface is the hair and scalp.

26. The method of claim 11, wherein X is the methanesulfonate ion.
